(19) United States
(12) Patent Application Publication
(10) Pub. No.: US 2024/0023495 A1
(43) Pub. Date: Jan. 25, 2024

(54) STORAGE CONTAINER, GROWTH AND/OR PROPAGATION STATION, CULTIVATION SYSTEM AND METHOD FOR CULTIVATING DEVELOPMENT MATERIAL

(71) Applicant: CAPSERO AG, Münchenstein (CH)

(72) Inventors: Benjamin RUTZ, Liestal (CH); Günter DÜRR, Seltisberg (CH); Fabio CIRILLO, Ormalingen (CH)

(21) Appl. No.: 18/023,788

(22) PCT Filed: Aug. 23, 2021
(86) PCT No.: PCT/EP2021/073275

§ 371 Date: Feb. 28, 2023

(30) Foreign Application Priority Data
Sep. 2, 2020 (EP) 20194195.2

(51) Int. Cl. A01G 9/26; B65D 81/22; B65D 81/20; B65D 65/40; B65D 81/30; B65D 81/38; B65D 85/52; B65D 88/74; A01G 7/02; A01G 9/24

(52) U.S. Cl. CPC A01G 9/26; B65D 81/22; B65D 81/2069; B65D 65/40; B65D 81/30; B65D 81/38; B65D 85/52; B65D 88/74; A01G 7/02; A01G 9/246; B65D 2565/388; B65D 2565/387

(57) ABSTRACT

A storage container includes an interior space and a substrate disposed therein, a biological living development material disposed in or on the substrate. The storage container is a sealed capsule to provide an aseptic atmosphere. The substrate is an absorbent material having a water absorption capacity in the dry state of at least 50 g water/cm³. The storage container has two end segments and a tubular middle segment. The substrate and the development material are arranged in the middle segment. At least a first one of the end segments is arranged opposite the middle segment. The substrate and the development material are arranged in the middle segment. At least a second one of the end segments is arranged opposite the middle segment and has a smaller wall thickness than the middle segment, has a wall material that is more thermally, physically, or chemically susceptible to attack than the middle segment, has a predetermined separation point.

### BACKGROUND AND SUMMARY OF THE INVENTION

Exemplary embodiments of the present invention relate to a storage container, a growth and/or propagation station, a cultivation system, and a method for cultivating development material

JP 4,979,976 B2 discloses an automated aseptic cultivation room. This technology has the disadvantage that, due to the technical installations, the room cannot be used maximally for planting and the planting material cannot be introduced aseptically or the room sterilized after the planting material has been introduced. In addition, commercially packaged incrusted seeds are available, but these are also not placed in an aseptic atmosphere and in a defined position of the package.

Furthermore, greenhouses are known from JP 4,979,976 B2, in which a harvest is carried out under aseptic conditions by means of harvesting robots. Further JP 4,979,976 B2 and WO2017/041757 A1 discloses accessible containers for the cultivation of plants or similar goods, which permits cultivation of seedlings from seeds, as well as the simultaneous raising of plant goods from seedlings and devices for their harvest. However, these do not have any provisions for aseptic processing after harvesting.

US 2015/027049, EP 3,398,429, and U.S. Pat. No. 5,375,372 disclose a transport container that permits aseptic cultivation of a product, but on the one hand makes use of adapters for the supply and removal of media, or on the other hand accomplishes this via permeable or semi-permeable openings or membranes to the outside. In the prior art, sections are also named which are intended to prevent an exchange of media or to draw off nutrients and liquid from a reservoir through conduits or similar openings.

Thus, no transport container is known from the prior art that permits aseptic transport under hermetic conditions and, after introduction into a cultivation room, can be sterilized on the one hand and is self-opening on the other. In addition, the cultivation of development material in the aforementioned prior art is only possible to a limited extent, depending on the stage of development, and does not permit the cultivation of high-growth or large-volume development material.

Based on the present state of the art, exemplary embodiment of the present invention provide a development material for cultivation in such a way that the development material is not contaminated during introduction, development, harvesting, and processing. The cultivation of the development material can be carried out in dependence on the development stage. The transport container of the development material takes up as little volume as possible in accordance with the development material. The development material can develop both inside and outside the transport container until harvesting under aseptic and optimal conditions corresponding to the development material, i.e., different climates. Furthermore, the development material should be provided in such a way that it can be transported in a protected manner, which already uses the transport period for cultivation, which can be stored well, and which can be expanded in a modular manner within the framework of farm management. In particular, this task arises the requirements of regulated industries, such as pharmaceuticals, cosmetics, and food supplements.

A storage container according to the invention comprises an interior space and a substrate arranged therein. The substrate forms the surface that allows growth or propagation of biological living developmental material. In particular, the substrate may allow rooting, i.e., root penetration or attachment. Porous materials, such as porous rock or a sponge material, or gel-like materials, such as hydrogels or gels of biological materials, are particularly well suited for this purpose. Particularly preferably, the development material has a high water absorption capacity. Instead of water, other liquid media, e.g., a nutrient solution, can of course also be absorbed. Nutrients can be contained in the substrate in liquid form, for example, but also in solid form, e.g., as salt.

The substrate can preferably extend over the entire width of the interior space and, according to the invention, is arranged stationarily on the wall of the storage container, e.g., by frictional connection or e.g., by support of the substrate on a net layer arranged perpendicular to the longitudinal axis of the storage container. Alternatively, or additionally, the substrate can also be formed in such a way that, without a support structure, the substrate is stationary by adhesion to the wall of the storage container and by cohesion in the substrate itself.

According to the invention, the substrate is formed as an absorbent material having a water absorption capacity in the dry state of at least 50 g water/cm³. The water absorption capacity may comprise both unbound water and unbound water, such as is present in the form of hydrogel. This can eliminate the need for additional water accumulation in the end segment of the storage container. Humidification of, for example, plant roots, does not vary with the filling level of water. The opening of the end segment of the storage container in an aseptic atmosphere is not hindered by outflowing water.

The absorbent material can have several components or be formed in several layers. For example, the absorbent material can be a sponge-like material, which may be held in position by an additional support structure. Alternatively, the absorbent material is a dimensionally stable sponge material possibly with additional incorporated nutrients. Fixing the substrate without losing its dimensional stability is advantageous, especially since this can prevent the formation of a reservoir, e.g., an accumulation of water, in the area of the end segment, which would make it difficult to open the storage container at the bottom end segment.

According to the invention, the storage container further comprises a biological living development material arranged in or on the substrate. The development material can preferably be a plant, for example in the form of a seed, a seedling, or a cloned seedling. However, fungi, fungal spores, algae, or other biological organisms may also be used as developmental material. Further organisms which fall under the term “development material” are mentioned in the following description.

Particularly in the case of plants, the skilled person would provide an open device, e.g., a flower pot or the like, for a storage container, since the plant may become moldy if stored for a longer period of time, a water supply is not possible and an adequate supply of nutrients is not otherwise ensured.

In summary, biological living development material may be formed as a plant, clusters of cells, seeds, synthetic seeds, or embryos, particularly as a cloned biological living being. In particular, the biological living development materials not exclusively tied to a volume to biological mass ratio.

However, contrary to this consideration, the storage container is designed according to the invention as a closed capsule, the storage container preferably having a tubular middle segment defining the longitudinal axis. The capsule is only intended to bridge the short period between the provision of the plant, e.g., a cloning process, and the insertion of the plant into an aseptic atmosphere of a container. The stay of the development material in the storage container in a closed state is therefore preferably less than 14 days, in particular less than 5 days. Provided that the storage container is in the aseptic atmosphere, the latter can be opened, allowing water supply and air exchange, and preventing mold growth.

The storage container thus ensures an aseptic atmosphere, preferably an aseptic according to ISO 11135, ISO 11137, ISO 17665-1, ISO 13408-1, EU GMP Annex 1 or US cGMP with a sterilization process target of a SAL of at least 10⁻⁵. Particularly preferably, the storage container consists of a dimensionally stable material that can be inserted into an opening of a storage rack.

According to the invention, the storage container has two end segments and a tubular middle segment, the substrate and the development material, in particular the plant, being arranged in the middle segment, and at least one of the end segments having a smaller wall thickness than the middle segment or having a dimensionally stable wall material that is more thermally, physically and/or chemically attackable than the middle segment. A thermal attackability may consist in a difference of the melting point of the respective wall materials. A physical attackability can be, for example, a better mechanical destructibility, e.g., by ultrasound or by mechanical vibration.

Alternatively, or in addition to the different wall thickness and/or the different destructibility, a predetermined separation point can also be provided between the middle segment and the end segment. The predetermined separation point can be a predetermined breaking point. It is also possible that a heating wire melts the material at this point (e.g., by inductive heating). In this case, the predetermined breaking point would be a predetermined melting point.

At least the middle segment of the storage container can preferably consist of a dimensionally stable wall material. This has particular advantages for holding and storage in a storage rack, as distinguished from a film bag or the like. In the context of the present invention, dimensionally stable means that the wall material, unlike a film, is not foldable, but forms a container, i.e., a shaped body. However, deformability of the wall, for example in the case of a plastic capsule, is possible within the scope of the present invention.

The storage container is also preferably designed in such a way that it cannot be autoclaved, but can be surface sterilized.

The substrate is preferably fixedly arranged in the region of the center segment. The substrate may comprise a porous material. The substrate may further comprise a water-soluble material incorporated into the absorbent material.

The storage container can be designed as a hermetically sealed capsule without additional connections for ventilation or medium supply or for filtration.

The storage container can be designed in such a way that it can be opened on both sides in an aseptic environment. For the insertion and positioning of the development material, the substrate is arranged in the center segment.

In order to ensure at least a gas exchange in the closed state of the storage container, at least one of the end segments can be formed from a gas-permeable material. In particular, the material can be formed as a gas-selective semi-permeable material, preferably CO₂, O₂, N₂ and/or CH₄-selective.

The storage container may advantageously have a space below the substrate as a gas-filled or vacuumed cavity, which may preferably serve to contain roots that have outgrown the substrate. This cavity is bounded by wall sections of the storage container and by the substrate. This means that no water accumulation is provided in this area.

The first of the end segments, which has a smaller wall thickness than the middle segment and/or has a wall material that is more susceptible to thermal, physical and/or chemical attack and/or has a predetermined breaking point, is preferably formed as a bottom end segment with respect to the biological living development material.

A wall of the storage container, in particular of the middle segment, can be made at least partially or completely of a transparent material, so that visual monitoring of the condition of the development material in the storage container is possible. Particularly preferably, the material can consist of a transparent, UV-impermeable, thermally insulating material that is at least partially gas-tight against the outside.

For a defined position of the storage container in an opening of a storage rack, the center segment can have at least one circumferentially distributed limit stop or several circumferentially distributed limit stops as an axial stop or a conical shape for the same purpose.

Alternatively, or additionally, the storage container may also have a locking mechanism to fix the storage container in a stationary and vibration-resistant manner. Preferably, such a limit stop and/or a locking mechanism is attached to the center segment, in particular to the lower half of the center segment.

Particularly preferably, the substrate is arranged as the only water reservoir within the storage container. In this case, the substrate has a water content of at least 30 g water/cm³ and is preferably positioned as a shaped body, in the middle segment of the storage container. This means that the substrate does not necessarily have to be filled with water up to its maximum absorption capacity, although this is preferred.

The storage container may also have an all-encompassing sterilizable surface that is sterilizable by suitable sterilization methods, such as UVC, ethanol, ozone, perchloroacetic acid, hydrogen peroxide, and so forth. Sterilization can be performed as CIP as well as WIP, wipe sterilization, immersion sterilization, exposure, or gassing. Particularly suitable for this purpose are materials of the storage container which are based on a metal, such as aluminum, iron, a thermoplastic, such as polyamide, polycarbonate, polyolefin, polyacrylic, polymethacrylic, halogenated ethylene, etc., and/or an elastomer, such as rubber, halogenated elastomers, silicones, etc., i.e., at least 50% of which consist of these.

Furthermore, according to the invention, there is a growth and/or propagation station for cultivating development material, wherein the growth and/or propagation station is designed as a large-capacity container, in particular as an ISO container, and wherein the large-capacity container has a device for generating and/or monitoring an aseptic internal atmosphere.

In particular, the device can be designed as a sensor-monitored distributor device. Such a distribution device can have inlets and outlets, actuators such as fans or pumps, and control devices such as valves or gas flaps. Further part of the distribution device is a sensor arrangement comprising one or more sensors for determining various parameters. Another part of the distribution device can be at least one control and/or evaluation unit, which receives measurement data from the sensors and, if required, adjusts the actuators or control devices to a setpoint based on the measurement data.

Thus, the aforementioned device is constructed and arranged to provide and monitor an aseptic internal atmosphere while setting, monitoring and/or maintaining optimal growth conditions based on various parameters.

The monitoring, adjustment, and/or maintenance of the parameters serve to ensure the growth conditions. They include the temperature, air pressure, humidity, and the gas exchange rate, which can be determined and adjusted by the device by means of sensors.

Optionally and especially preferably, the parameters air pressure, light intensity, fertilizer amount, fertilizer composition, pH, and/or conductance can also serve for an even better realization of the monitoring, adjustment, and/or maintenance of the parameters.

In particular, the monitoring and adjustment of the gas exchange rate parameter is beneficial to create a controlled, hermetic atmosphere while optimizing energy efficiency.

Corresponding sensors for fulfilling this task are known to the person skilled in the art. Humidity sensors, pressure sensors, ion-selective electrodes, flow sensors, conductivity sensors and the like can be used, among others.

Further parameters which the device additionally determines, monitors, and/or adjusts by sensors, individually or in combination, are SAL level, partial gas pressure, fertilizer temperature, wind strength, wind direction, sound level, and/or sound frequency sequence. SAL is the sterility assurance level which can be determined by combining several measured values.

The monitored and adjustable partial gas pressure can be the partial gas pressure of individual or in combination of the following gases: CO₂, O₂, N₂, He, Ar, O₃, CO, CH₄, ethane, ethene, ethyne, and/or terpenes.

Specifically for terpenes, it may be the concentration of one or more of the following terpenes: Hemiterpenes, Monoterpenes, Sesquiterpenes, Diterpenes, Sesterterpenes, Triterpenes, Tetraterpenes, Polyterpenes, and/or Terpenoids.

Advantageously, the growth and/or propagation station has a grow room with a supply and/or discharge of a CIP medium, in particular UV-C radiation, gases, such as ozone, ethylene oxide, propylene oxide, or a nebulized liquid medium, such as hydrogen peroxide, inorganic acids, or inorganic bases. For gases and mists, it is also preferred that a distribution system be provided to decontaminate the entire interior. In particular, this can be done with ventilation or pressure-fed nebulized media. In particular, ventilation distribution is advantageous for gases and spray nozzles for fluids.

Monitoring of successful decontamination is measured by appropriate sensor technology, such as gas sensors to determine gas concentration, radiation sensors to determine photon flux, pH sensors to determine acid or base strength, and/or ion-selective electrons to determine ion strength. In conjunction with concentration, exposure time must be measured to confirm successful decontamination.

During decontamination, ozone is advantageously used, which is formed by arc discharge or by UV-C on atmospheric oxygen and is introduced into the grow room until the concentration and/or the exposure time has been reached.

After successful decontamination, the excess CIP medium is removed from the grow room so as not to damage the development material. Suitable measures for this can be aeration with fresh air or process gas, such as nitrogen, carbon dioxide, argon and/or traces thereof in air, downwashing, or titration. It is advantageous to wash down with water, followed by neutralization.

Furthermore, the large-capacity container can have at least one storage rack for arranging one or preferably a plurality of storage containers according to the invention. Preferably, the storage rack or racks is or are arranged movably, in particular displaceably, relative to the container wall. For this purpose, the storage racks can be mounted on roller bearings, on compressed air bearings and/or on plain bearings.

Further preferably, the large-capacity container according to the invention may comprise at least one, more or preferably all of the following devices:
a light source for irradiation of the development material;
a pump for media transport, including a nutrient-containing medium and/or a sterilization medium;
a sensor to monitor the health of the developing good;
a control and/or evaluation unit for controlling a control circuit for adapting the indoor atmosphere to the changes detected by the sensor;
a water supply and/or treatment unit;
an air conditioner;
a nutrient container;
a power supply and/or management unit;
a mixing chamber; and/or
a communication module for data exchange with an external device.

Individual devices, in particular electronic devices, can be arranged in a service area spatially separated from the grow room, which is accessible from the outside and is not subject to the aseptic atmosphere.

Media and/or signal or power lines from this service area into the grow room are preferably cast in a media-tight manner in a partition wall, so that the internal atmosphere cannot penetrate into the service room and, conversely, the aseptic conditions in the grow room are maintained even when the service area is accessed. Further advantageous embodiments of the growth and/or propagation station result from the following description and the figures.

Further according to the invention, a cultivation system comprising a growth and/or propagation station and a maintenance unit, in particular a harvesting unit, wherein the maintenance unit is connected to the growth and/or propagation unit for transferring development material or machinery between the two units, and wherein the maintenance unit has the aseptic internal atmosphere.

The maintenance unit can be part of the container which also has the grow room or, especially preferred for a larger modular arrangement, the maintenance unit can be arranged in a separate container. An airlock can be arranged between two containers and decontaminated with suitable measures, such as negative pressure, especially vacuum, or as mentioned above one or more CIP media. By opening both containers, the airlock can be flooded with the aseptic internal atmosphere. However, this represents only one variation of coupling the two containers. A double door on one or both of the two containers can also represent a safety device for coupling the containers. In this case, the containers have suitable coupling devices to firmly connect the two containers in adjacent positions, preferably in a medium-tight manner.

Further according to the invention is a method for cultivating development material, which comprises at least the following steps:
a) Providing a storage container according to the invention,
b) Placement of a developing product into the storage container under aseptic conditions.
c) Inserting the storage container into a storage rack of a growth and/or propagation station, in particular a growth and/or propagation station according to the invention, and opening the storage container at least on both ends.
d) Monitor growth and/or propagation of development material in the aseptic internal atmosphere, until detection of a growth and/or propagation stage for harvesting;
The growth or propagation stage can be determined by optical analysis, e.g., of the size of the plant, the spread of a bacterial strain, or the outgassing of organic matter, and can be compared to a target value. However, other measurement methods are also possible within the scope of the present invention.
e) Use of a maintenance unit while maintaining the aseptic atmosphere with recovery of a valuable material, in particular a biological material containing an active substance.

Next, a maintenance unit, preferably a maintenance unit according to the invention, is used to recover the valuable material.

Preferably, sterilization takes place in step c), preferably after insertion of the storage container, preferably as part of a CIP process. This implies that the storage container withstands the sterilization conditions at least during sterilization and thus protects the development material.

Further advantageous embodiments of the invention, both of the method and of the devices, are described below.

A first process step of the procedure can be, among others, the introduction of development material into the storage container together with substances that can influence the growth.

The development material may be individual seedlings or some form of developable, especially living, biomass.

The storage container can serve as storage, manipulation, or transport protection for the development product, especially in the case of ground, water, or air transport.

The storage container can be sterilized in the process and can be opened automatically, preferably after insertion into trays of the storage rack and after sterilization of the internal atmosphere.

Furthermore, the storage container can be stably anchored in the trays and can be clearly identified by a marking placed on the storage container.

Furthermore, a single growth and/or propagation station or several such stations combined to form a so-called farm may have a service area, hereinafter also referred to as process room, and a maintenance unit.

The growth and/or propagation station or farm may allow continuous aseptic cultivation of biomass to be cultured, called development material, without requiring or allowing a human to enter the system.

The growth and/or propagation stations are stackable and have stops for stacking typical of ISO containers, especially in the corner areas.

The growth and/or propagation station or farm may have indoor atmospheric conditions in the unit (referred to as indoor atmosphere), which may preferably be provided according to the needs of the development material by
i) the internal atmosphere conditions in the unit are set up as closed-loop, and that
ii) the atmospheric conditions can be controlled and meet the aseptic criteria of a clean room according to ISO and/or GMP,
iii) the temperature, humidity, nutrient composition, pressure, electromagnetic field, air flow, atmospheric composition, pH, light intensity, light spectrum, and/or day/night cycle can be provided to the development material as required,
iv) the conditions in the indoor atmosphere are measured and recorded just in time,
v) the recorded data is used locally and/or centrally for analysis and interpretation, processed and used to control further processes.

The growing and/or propagation station or farm may have the mass and transport characteristics of standard containers (ISO containers) that can be transported by truck or ship.

The growth and/or propagation station or farm may include at least one power supply and a communication module, particularly a radio module, for operation.

The unit with a storage rack or shelving system optimized for mounting space can be formed from at least two shelves that can be moved parallel to each other, each with a supply system integrated in the shelves.

All liquid media, partially also the gaseous media and the energy, the sensors and their connection can be led in the cavities of the rack systems.

The rack system allows for the delivery of nutrients to the crop for cultivation. In particular, the nutrients can be conveyed both aeroponic and hydroponic, or a mixture of these, to the cultivation material.

Aeroponic cultivation can preferably be accomplished from a low-pressure system by nebulization using piezoelectric elements.

Hydroponic cultivation can be accomplished by low-pressure system, especially by nutrient film technology.

The individual shelves can contain one or more trays for holding storage containers, which can hold the storage container or a plurality of storage containers. The trays are modular, reusable, cleanable and sterilizable, which are provided with the possibility of fixed anchorage of the storage container.

The maintenance unit allows the aseptic transfer of a development material from the growth and/or propagation station to the maintenance unit, in particular in the form of a harvesting unit.

The maintenance unit is preferably movable in horizontal and vertical direction,
(a) for traversing in the vertical direction, a scissor lift can be used,
(b) for traversing in horizontal direction, a Mecanum drive can be used,

### BRIEF DESCRIPTION OF THE DRAWING FIGURES

Individual embodiments of the invention are described in more detail below with reference to the accompanying figures. The figures merely show preferred embodiment variants and are not limiting to the subject matter of the present invention. However, the person skilled in the art will also apply individual elements of the respective embodiments to further embodiments, so that these are disclosed not only in the context of the specific embodiment. Showing:

FIG. 1 Sectional side view through a storage container for living biomass, in particular biomass capable of growth or reproduction;

FIG. 2 Sectional view in the longitudinal direction of the growth and/or propagation station;

FIG. 3 Rear view of the growth and/or propagation station;

FIG. 4 Sectional side view through a maintenance station for connection to the growth and/or propagation station;

FIG. 5 Sectional side view through a first maintenance unit for connection to the growth and/or propagation station; and

FIG. 6 Sectional side view through a second maintenance unit for connection to the growth and/or propagation station.

### DETAILED DESCRIPTION

By means of known cloning or other reproduction or production methods, biomass is produced, which is suitable as a starting product and which reproduces and/or develops, e.g., grows, in a subsequent growth phase. The growth phase is hereinafter also referred to as maturation.

The biomass may belong to the group of procariotes, as well as to the group of eucariotes. In particular, the biomass may be produced by means of somatic embryogenesis, zygotic embryogenesis, and/or apomixis and subgroups thereof via a method characterized by the ability to maintain aseptic conditions.

In the case of cloning the starting cells can be derived from the meristem, the simple permanent and/or the complex permanent tissue. Furthermore, stem cells, spores, sperm, oocytes and/or semen can be used.

Further, the biomass can be preserved by suitable chemical, biological, and/or physical measures such as hormones, toxins, enzymes, and/or by cooling, freezing, or drying. In the following, the biomass is called development material, because the biomass obtained is to be supplied to its development to the harvestable form in the process steps I, II and III described here.

In a first step, the development material is placed under aseptic conditions in a package, hereinafter referred to as a storage container.

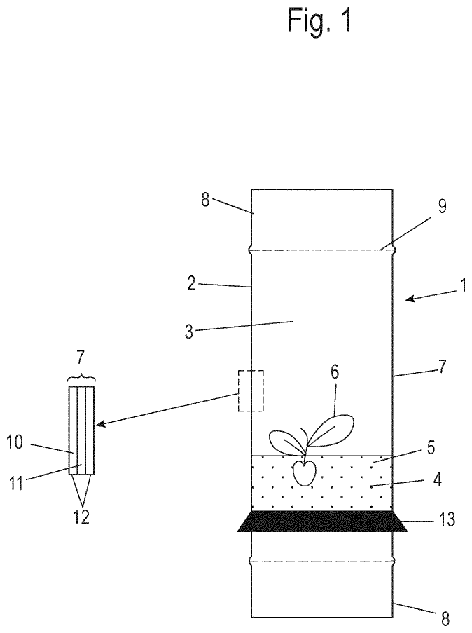

FIG. 1 shows a storage container 1 for an aseptic storage container which is hermetically sealed from the environment. For this purpose, in the embodiment variant, the storage container has, in the region of a middle segment 2, an interior space 3 in which a substrate 4 is arranged for the provision and/or storage of nutrients 5 for a development material 6 arranged in the substrate 4 and grown under aseptic conditions, in particular a plant seed, plant seedling or a plant germ and/or a fungal spore and/or alga or the like. Such a substrate may comprise, inter alia, water or hydrate-containing gel, as well as various salts, hormones, vitamins, carbohydrates, chelates, and/or amino acids that further support plant growth.

The interior 3 is bounded by a wall 7 which extends circumferentially around the substrate 4 in the form of a tube. The center segment 2 has an end segment 8 on both sides, which closes off the interior 2 at the end. The end segments 8 have a detachable connection, e.g., a sealed mechanical interface and/or a predetermined breaking point 9, at the transition area to the center segment 2.

The mechanical interface 9 can be designed as a frictional connection of the end segment with the middle segment, e.g., with a circumferential seal, whereby the end and middle segments are in each case connected to one another via a mechanical connection mechanism, e.g., a latching mechanism. Alternatively, or additionally, a material-locking connection can also be provided, as is the case with a predetermined breaking point, or an insulating and/or adhesive connection, as is the case with a fusible seal, for example.

Depending on the variant, the interface may also have a film hinge which, after the mechanical connection mechanism is released, connects the respective end segment to the center segment 2 as a lid-like embodiment.

Alternatively, or additionally, the storage container 1 may have a water and/or contamination repellent protective coating 10, for example a wax coating. The coating enables surface sterilizability as part of a CIP process or any other sterilization process and/or maintains the aseptic barrier. A CIP process is understood here to be a cleaning and/or sterilization process in which cleaning in place or sterilization in place can be performed. A CIP process is understood to include cleaning, rinsing, washing, and sterilization.

For this purpose, the wall 7 of the storage container 1 has a barrier layer 11, preferably made of glass, polyolefin, polyamide, halogenated polyvinylene, therephthalates, and/or EVOH, the diffusion barrier layer preferably occupying at least 2% of the wall thickness, preferably between 10-100% of the wall thickness. Furthermore, the diffusion barrier layer can be arranged between two support layers 12. This can be any transparent material so that the condition of the plants within the storage container is visually recognizable.

The wall 7 of the storage container 1 additionally has UV protection in the form of the material and/or material additives, such as polymer additives of the series of benzotriazoles, triazines, acrylates, phenones, and/or HALS and/or as a barrier layer 11 preferably of UV-inhibiting material, such as pigmented and/or opaque polymers,

The storage container 1 has a preferred dimensional stability such that transport of the storage container and the goods contained therein is possible by ground, water, and air. In particular, the storage container has a dimensional stability of a pressure difference of at least 255 hPa, more preferably 500 hPa.

Furthermore, the storage container 1 has at least one limiting stop 14 protruding radially from the center segment 2 and limits the insertion depth of the storage container 1 into a storage rack 14 in a growth and/or propagation station 100.

Due to the mechanical interface and/or the predetermined breaking point 9, the storage container 1 can be opened at a defined position, preferably automatically. Thus, the opening of the storage container 1 at both ends can take place without human intervention in the aseptic atmosphere of a growth and/or propagation station 100 described below and also according to the invention.

In particular, the storage container 1 is shaped in such a way that it can be automatically inserted into the opening of the storage rack 14 provided for the ripening of the development material 6 at the place of ripening. The openings for ripening in the storage rack 14 are hereinafter referred to as the setting place. The phase between insertion of the development material 6 into the storage container 1 up to the insertion of the storage container into the setting place is hereinafter called process step II.

The setting place is a place in a growth and propagation station 100, hereinafter also referred to as a grow room, where the storage container 1 remains during maturation until harvesting and where the development material 6 is kept stable by means of the storage container 1.

The storage container 1 according to the invention is provided with the development material 6 placed on the aforementioned substrate 4, which contains a depot of substances capable of stopping, inhibiting, retarding, slowing down, accelerating, promoting, and/or allowing the growth or multiplication of the development material 6.

The storage container 1 is designed to provide a sterile barrier between the environment outside the growth and propagation station 100 and the development material 6. The storage container 1 provides this sterile barrier both during the packaging of the development material 6 and during its storage, transport, handling, and introduction to the setting place in the growth and propagation station 100. The storage container 6 is sterilizable, in particular after their introduction into the grow room of the growth and propagation station 100, whether by means of liquids, radiation, gases or a mixture of the aforementioned variants. The storage container 1 protects the development material 6 during this sterilization process.

After sterilization, the storage container 1 can be opened by an opening procedure that can be automated and/or controlled.

Variants of this opening procedure includes
I. mechanical processes such as bursting, blasting, cutting, dissolving, penetrating, unscrewing, (un)twisting, pressing, sound, ultrasound, vibration, tapping, hitting, perforating; and/or
II. by degradation or decomposition, in particular by thermal and/or chemical means; and/or
III. through fermentation; and/or
IV. by energy input, such as direct current, alternating current, electromagnetic radiation, magnetism, laser; and/or
V. a mixture thereof of two or more of the foregoing processes.

As previously described, the storage container 1 contains substances for supplying the development material 6 during storage, transport, and introduction into the growth and/or propagation station 100. These may include, but are not limited to, water, nutrients, and other media or substances. In particular, the substances may either interrupt, inhibit, slow down, promote, accelerate, and/or continue the development cycle.

The wall of the storage container 1 may also allow gas exchange while maintaining the sterility of the development material 6. Preferably, as an alternative to or in addition to the diffusion barrier layer, the wall of the storage container may provide at least one wall segment that permits gas diffusion but is diffusion-tight with respect to liquids. Particularly preferably, a membrane can be incorporated into the wall, which allows gas to escape on one side, but not liquids. In particular, a membrane can be incorporated into the wall which allows specific gases or gas mixtures to exit or enter in a targeted manner.

On the outside, the storage container 1 can have a marking showing an identification of the development material 6 and the position or the setting place of the storage container 1, as well as possibly further information. The marking, and consequently with it the development material 7, can preferably be formed as QR code, bar code, data matrix code, dot matrix, symbols, color or colors, patterns, RFID, or a mixture of these above-mentioned variants, so that the development material 7 is identifiable and traceable.

The invention further relates to a cultivation system comprising at least one growth and/or propagation station 100 or a plurality of assembled modular grow rooms, hereinafter referred to as farm 200, in which process step III takes place

Each individual growth and/or propagation station 100 may be in the form of a container having standard overseas container dimensions, i.e., a large-capacity container as defined in ISO 668, as in effect at the priority date of the present invention.

The container can be transported in particular by truck or ship, can be stacked and/or bolted by means of usual devices according to ISO 668 and thus a connection of several growing and/or propagation stations 100 and possibly of further stations to a farm 200 becomes possible.

The growth and/or propagation station 100 is thermally separated from the outside world by an inner insulation 15 and can provide a desired climate. In particular, the inner insulation 15 is made of polystyrene, polyurethane, cellulose, mineral materials, glass or foams, fibers, or wools thereof. Thus, the development material 6 is provided with its own hemisphere, further referred to as internal atmosphere, which can provide for each growing and/or propagation station 100 or for the whole farm according to the conditions required by the development material 6 to the development material 6 over the various ripening cycles.

The growth and/or propagation station 100 further comprises a sluice-like installation and/or docking point 16 and optionally, in the extension to a processing system, a maintenance unit 17 at a first end side of the growth and/or propagation station 100. By means of the maintenance unit 17, hereinafter referred to as maintenance unit 300, the maintenance of the growth and/or propagation station 100 and/or the harvesting can be performed in a contamination-free and sterile manner. In this regard, the maintenance unit 300 may have, among other things, a double door 18 that enables the airlock-like installation. In this regard, harvesting is performed in process step IV.

During process steps II-IV, the growth and/or propagation station 100 should not be entered by humans. This is necessary in order to provide the development material 6 with an aseptic internal atmosphere and, as a consequence, to avoid the use of pesticides for the treatment of the development material 6.

A media supply can be provided via a second end face, the technical side 19, of the growth and/or propagation station 100, which is preferably in the form of a container, and can thus be accessible for maintenance and servicing work from the outside, in particular also during process step III. All materials and media required for the growth and propagation of the development material 6 can be supplied and/or removed in each case via the technical side 19 of the growth and/or propagation station 100. Optionally and particularly preferably, maintenance cycles may be provided for this supply and/or removal in order to maintain control and asepsis within the grow room.

The growth and/or propagation station 100 comprises at least one dedicated power supply and management unit 20. The energy supply and management unit 20 comprises at least one dedicated communication module 21. In order to permanently provide power important for the development material 6, the power supply and management unit 20 may further have an uninterruptible power supply 22.

In order to allow aseptic production of the development material 6, in which the development material 6 is completely hermetically separated from the outside world, the technical design of the growth and/or propagation station 100 should be such that clean room criteria according to ISO and/or GMP exist for biological contamination within the grow room for process step II to IV.

Further, a light source 23 may be provided to provide calibrateable light conditions to the development material 6, i.e., to provide the development material 6 with any other atmospheric conditions such as the day/night cycle or a required variable light spectrum and light intensity. In particular, a constant light intensity can be provided to the development material 6 during the entire growth cycle.

Furthermore, the growth and/or propagation station 100 has a humidification and/or a ventilation unit, hereinafter referred to as air conditioning unit 24, to provide the required humidity, atmospheric composition, and winds to the development material via preferably ventilation slots 25.

Furthermore, the growth and/or propagation station 100 comprises a nutrient or irrigation system 26 in order to provide the development material 6 with the required coordinated nutrients for the entire growth cycle and to make them available at the points required by the development material 6, for example at the root or leaf structure. In particular, it is advantageous to supply the nutrients in an irrigation system 26 in such a way that the constituents or the composition of the nutrient medium is known. This can be realized by disinfecting, deionizing and/or removing heavy metals from water by a water treatment 27. This treated water can be temporarily stored in a reservoir 28 or recycled in order to be returned to the nutrient medium or the irrigation system 26.

Aeroponic cultivation is characterized by wetting roots with aerosol of a solution of nutrients and water. To implement this technology, the growth and/or propagation station 100 may have, for example, a low-pressure system. Alternatively, a high pressure system or an ultrasonic atomizer may be used. In the preferred case of a low-pressure system, nebulization of the solution can be performed using piezoelectric elements.

Alternatively, or additionally, hydroponic cultivation by means of NFT (nutrient film technique) can also be made possible. In this case, the development material is placed in net pots or similar, which allow the roots of plants or mycelium of fungi or other parts of the development material to protrude from the pot and project into a liquid-carrying channel in which a nutrient solution is guided. This channel is slightly slanted and thus provides a flow direction for the nutrient solution. The nutrient solution can then be discharged via discharge openings and collected in a collection area below the trough and, if necessary, recirculated, preferably pumped, back into the trough. An air space is arranged between the nutrient solution and the pot, which must be overcome or bridged by the parts of the development material in order to reach the nutrient solution.

The nutrient medium is mixed from water that has previously passed through the water treatment 27 and/or has been conveyed from the reservoir 28 by means of actuators 29 and/or gravitationally and the various nutrients, which are advantageously stored in nutrient containers 30. Advantageously, the nutrients from the nutrient containers 30 are mixed passively and/or actively by a mixing chamber 31. In this way, gradients, pulses, steps, or changes of nutrient medium compositions can be realized, which, coordinated with the growth cycle of the development material 6, allow the development of the development material 6 to be influenced.

In addition, the growth and/or propagation station 100 has a closed loop control system with a control and/or evaluation unit 32 and a plurality of actuators 29 and sensors 33. The control circuit detects the concentrations of the substances both in the internal atmosphere and in the supplied nutrient media, and can analyze changes in the internal atmosphere. Then, by controlling and/or regulating the actuators 29 of the control loop, the control and/or evaluation unit 32 enables an adapted inner atmosphere and/or nutrient media to be provided to the development material 6 based on the analyzed data. This can be done promptly or just in time, so that the preferred time period between the measurement and the adaptation is less than 30 min, preferably less than 15 min.

Advantageously in the context of the present invention, the aforementioned control loop is part of the growth and/or propagation station 100. The control loop can advantageously be designed as a closed control loop. It may comprise a control and/or evaluation unit 32 for its control. The control takes place, in particular, after the evaluation of sensor signals by corresponding sensors 33 within the growth and/or propagation station 100. The control and/or evaluation unit 32 and preferably also the entire control loop is preferably remotely controllable and particularly preferably bidirectionally controllable from an external device via a communication module 21. A more complex analysis of measurement data can be performed by the external device. Data storage may in particular be performed by the external device. The term “external device” in this context also includes an IT infrastructure, such as a cloud or a neural network.

Data transmission, preferably bidirectional data transmission, can take place between the control loop and the external device. In addition, an IT infrastructure in the form of the artificial neural network can also be used to control the control loop, which can, for example, recognize tendencies from the sensor data and draw conclusions about the overall state of the development material 6 by means of corresponding analysis logarithms. At this point, artificially generated swarm intelligence can thus be used to analyze a large amount of sensor data. The data transmission between the control loop and the external device is preferably carried out via a communication module 21 that transmits and/or receives via GSM, WiFi, LoRa, and/or Bluetooth.

The control loop is characterized in that the collected data from the sensors 33 are logged and used by an evaluation corresponding to the development material 6 to maintain, restore and/or bring about conditions. Advantageously, these conditions may be that a desired or required internal atmosphere or a change in nutrient composition is triggered. This can be applied in real time or in a time-delayed or so-called time-shift process. The control loop can use data from all grow rooms and all farms 200 for the analysis, both simultaneously and over a period of time. The data can be used for the purpose of an analysis of the growth phases, the state of health, or the maximization and/or minimization of parameters of the development material 6, in particular for the determination of limit and/or target values.

In addition, external literature, analyses, or other data may be supplemented, verified, or made plausible for analysis. Data may be drawn upon by methods of mathematical analysis, interpretation, causality, sense-making, decision-making, and/or forecasting. The analyses may be refined, supplemented, extended, and/or completed by statistical methods. In particular, statistical methods such as error analysis, ANOVA, series expansions, maxima, minima, differential, integral, and/or convergence analyses are appropriate.

The analysis, management, storage, archiving, and backup of the data may be performed remotely from the growth and/or propagation station 100 or from the farm 200 in the external device and makes use of common transmission protocols, techniques, and infrastructure, hereinafter referred to as data processing.

In this context, the data processing is characterized by the fact that bidirectional communication with the growth and/or propagation station 100 and/or the farm 200 is possible and that the data are (centrally) utilized by algorithms using AI (artificial intelligence), machine learning, or supervised machine learning, processed and the knowledge gained therefrom is used for the cultivation of the development material 6.

Further, as previously mentioned, the growth and/or propagation station 100 comprises storage racks 14 or shelves for storing the storage containers 1, particularly in the embodiment of a storage container. The storage rack 14 may be arranged to be movable within the grow room. Optionally, the respective storage rack 14 may also be movable into a connected maintenance unit 300. An advantageous variant of such a storage rack may be a roller storage rack, wherein the rollers are guided on a guide, for example a guide rail on the floor or ceiling of the growth and/or propagation station 100.

For cleaning and/or for reloading the storage racks 14 with trays 34 and/or storage containers 1, a respective storage rack 14 can be moved out of the respective grow room.

In this context, the storage racks 14 are preferably rack systems containing all the connections and/or installations necessary for supplying the development material 6 with media. These media are preferably nutrients, water, gases, wind, and/or possibly other substances influencing growth or reproduction. Necessary installations and/or connections may preferably be designed to be individual to the setting place and may include, for example, spray nozzles, supply and/or discharge lines, distribution systems, electrical signal lines, power supply lines, radio modules individual to the setting place, and sensors 33 and/or actuators 29 individual to the setting place. Nutrient containers 30 for nutrients and other media can be provided on a setting place-individual or storage rack-individual basis, which is particularly advantageous insofar as different development material 6, e.g., different plant varieties, is arranged in different setting places or storage racks.

A respective storage rack 14 can be constructed in several parts and preferably have insertable trays 34 and/or collecting trays with one or more receiving openings for one or more storage containers 1. The trays 34 can be sterilized separately or, if necessary, also discarded as single-use elements and exchanged for new trays 34 when used again.

Provided that only a single variety of development material 6 is present in a growth and/or propagation station 100, a single reservoir 28 and/or nutrient container may also be provided per growth and/or propagation station 100 or even only per processing system 400.

The storage racks 14 may further include installations necessary for disposal of the spent media. The preceding explanations regarding the reservoir 28 and nutrient tank 30 are to be applied accordingly to the collection tanks for residual medium.

Advantageously, the trays 34 are modular in design, whereby the development material 6 can preferably be provided with nine or more setting places per tray 34 for secure and controlled holding of the development material 6 during cultivation. The trays 34 further allow for efficient harvesting, as preferably a collection device for the roots or the part of the development material 6 facing the nutrient medium is attached to the tray 34. This can be, for example, a net, a bag, or/and a tub.

The movable storage racks 14 are installed in such a way that no circulation areas are necessary within the interior atmosphere, thus maximizing the use of the grow room for storing the development material 6.

Multiple growing and/or propagation stations 100 may be combined to form larger units called farms 200. Farms 200, i.e., larger units consisting of multiple individual grow rooms of any number, may be operated from a single maintenance unit 300, e.g., a separate container, for their maintenance, harvesting, and supply.

However, it is also possible to provide only one growth and/or propagation station 100 and also to arrange the maintenance unit 300 adjacent to the growth and/or propagation station 100 within a single container, preferably adjacent to the grow room.

The maintenance unit 300, the processing unit 500, and the growth and/or propagation station 100 thus form a processing system 400, which ensures growth/propagation of the development material 6 as well as harvesting, processing and packaging of the harvested development material 6 under aseptic and, in particular, hermetic conditions from the moment of insertion and sealing of the development material 6, e.g., as seeds in the storage container 1. In this regard, the processing system is distributed to one or more large-capacity containers or containers, which ensure the maintenance of a hermetic internal atmosphere at least for growth and propagation, but preferably also for harvesting, processing in a processing unit 500 and/or packaging.

For example, medicinal plants or the like can be grown under sterile conditions. The complete processing system 400 or at least the growth and propagation station 100, and particularly preferably each container of the processing system 400, thereby has inlets and/or outlets for the introduction of a cleaning medium, preferably a CIP medium, e.g., steam or the like—so that the system is sterilized for reuse after use.

The maintenance unit 300 allows manipulation of the development material 6 and monitoring of the development material 6 by means of automated systems, such as robotic systems and/or a time-delayed harvesting of the development material 6, thus allowing manipulation from the outside without contaminating the aseptic internal atmosphere. In this regard, one or more data sets relating to the type of development material 6 and the desired growth stage (e.g., plant size, leaf size, fruit size, ripeness of a fruit, fungal size, bacterial count, etc.) for initiating a harvesting process are stored on a data memory 35 of a control and evaluation unit 32. When a set point or combination of multiple set points corresponding to the growth stage is reached, the control and/or evaluation device 32 may initiate the harvesting process.

This may comprise, among other things, the activation of a harvesting machine, in particular a harvesting robot, or possibly also initially the hermetic coupling of a maintenance unit 300 to the growth and/or propagation station 100, as well as the subsequent movement of the harvesting machine, in particular the harvesting robot, into the growth and/or propagation station 100, or alternatively a movement of a storage rack 14 from the growth and/or propagation station 100 into the connected maintenance unit 300.

Alternatively, the opening device can also be arranged only in the growth and/or propagation station 100, e.g., as part of a movable robot, which is not associated with a storage rack 14. This solution is less preferred, however, since the time required to open each storage container increases with this solution.

The maintenance unit 300 may include bearing or transport elements, e.g., bearing rollers, for vertical and lateral maneuvering. For example, a lifting mechanism, particularly a lifting mechanism, and/or mecanum drive devices, e.g., mecanum wheels, may be provided as part of the maintenance unit. A single maintenance unit 300 may serve many grow rooms, process units 500, and/or farm 200.

The maintenance unit 300 may be used to maintain a single growth and/or propagation station 100, process unit 500, or farm 200. The maintenance unit 300 may harvest and process the development material 6, such as drying, crushing, separating, tempering, packaging, distilling, extracting, cleaning, photographing, and/or analyzing. One or more of these steps may be performed individually or in combination in process step III. For this purpose, the maintenance unit 300, i.e., the container may comprise, individually or together, a harvesting device, a drying device, a comminution device, e.g., a chopper and/or a mill, a filtration and/or centrifugation device, a temperature control device, e.g., a blast freezing device, a packaging device, a distillation device, an extraction device, a purification module, a photo or video apparatus, and/or an analytical device, preferably an HPLC, GC (gas chromatography), UV-Vis, Raman, and/or IR analytical device. Newer devices, e.g., process photometers, also allow in-line measurement in the processing process and can be applied in the present case.

Advantageously, the above-mentioned processes are outsourced to a separate container, i.e., process unit 500, and the maintenance unit 300 supplies the process unit 500 with harvested development material 6 from the growth and propagation station 100 via the respective docking points 16 at the growth and propagation station 100, the maintenance unit 300 and the process unit 500, in order to ensure continuous, contamination-free processing of a development material 6.

The maintenance unit 300 may be adapted to the atmospheric conditions and needs of the growth and/or propagation station 100 for performing process step III and/or IV. Further, the maintenance unit 300 may include a personnel lock to allow personnel to enter and exit under clean room or clean room-like conditions or to facilitate docking of additional units, e.g., a process unit 500. The process step III and/or IV in the maintenance unit 300 can be performed manually, semi-manually or automated. Due to the constant atmospheric and/or microbial conditions during the production of the development material 6 up to the process step IV, a to a high degree constant quality and homogeneity of the harvestable development material 6 and its processing can be achieved. The maintenance unit 300 and/or the process unit 500 may comprise a storage space for non-processed and/or processed goods, which may be temperature controlled, atmospherically controlled and/or placed under inert gas to protect the development material 6 from undesirable influences of the environment outside the container. Warehouse management may be automated or manual by lifts, pater-noster lift or similar system.

The embodiments shown in the figures are discussed in more detail below. FIG. 1 shows a storage container 1 in the form of a capsule. The storage container 1 has a wall 7 and has a middle segment 2 in the form of a tubular region, a first end segment 8 designed as an upper cap and a second end segment 8 designed as a lower cap. The upper cap or the first end segment can be designed to be gas-permeable. In the tubular region, i.e., the middle segment 2, there is a matrix or substrate 4 with nutrients 5 in which the development material 6 is arranged. Between the middle segment 2 and the end segments 8, mechanical interfaces and/or predetermined breaking points 9 are formed in each case. The mechanical interface 9 may comprise, for example, a latching mechanism. Further, at least one limit stop 13 is provided as a holding device circumferentially distributed on the wall 7. Circumferentially distributed in this case means several individual segments distributed around the circumference at a height of the center segment or a single segment extending fully at a height, which represents an axial stop and limits the insertion depth of the center segment 2 in an opening.

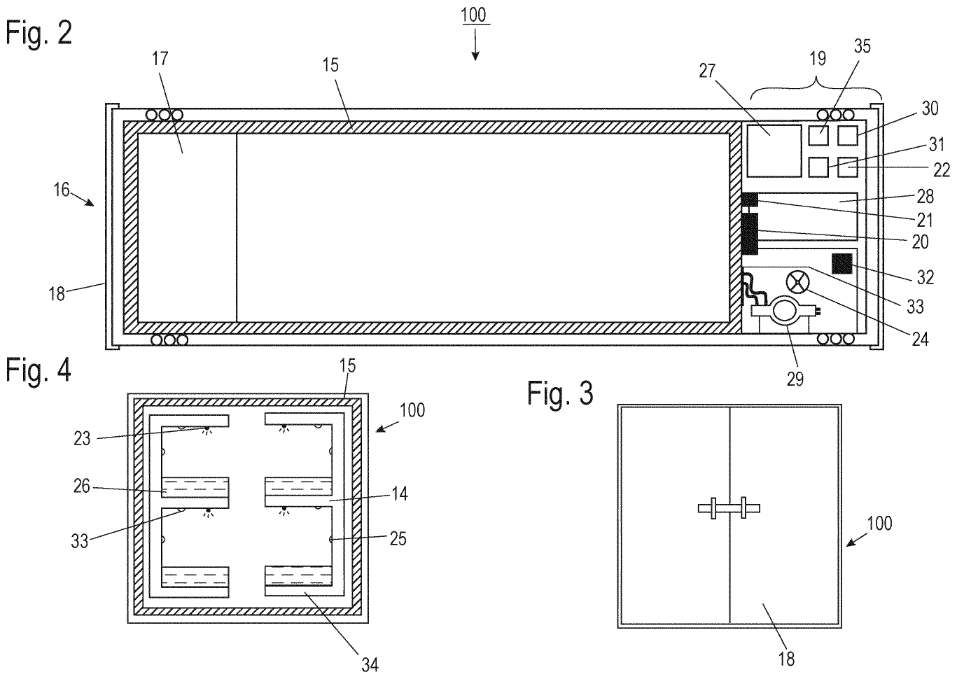

FIGS. 2-4 show a growth and propagation station 100 in the form of a container, which has an outer shell with devices that allow a force-fit connection to other containers or support devices according to ISO 668, an inner insulation 15, a supply unit 20, and a docking station 16 with double doors 18. Within the container, there may be an arrangement of one or preferably more storage racks 14 or a racking system that includes sensors 33, an irrigation system 26, a ventilation system or at least parts thereof, such as ventilation slots 25, and the light source 23. The supply unit 20 may each include one or more actuators 29, such as pumps and/or valves, sensors 33, a control and evaluation unit 32, an air conditioning unit 24, a water treatment unit 27, a reservoir 28, nutrient tanks 30, mixing chambers 31, an energy supply and management unit 20, and a communication module 21, preferably in the embodiment as a radio module. The container may be connected together to form a farm 200.

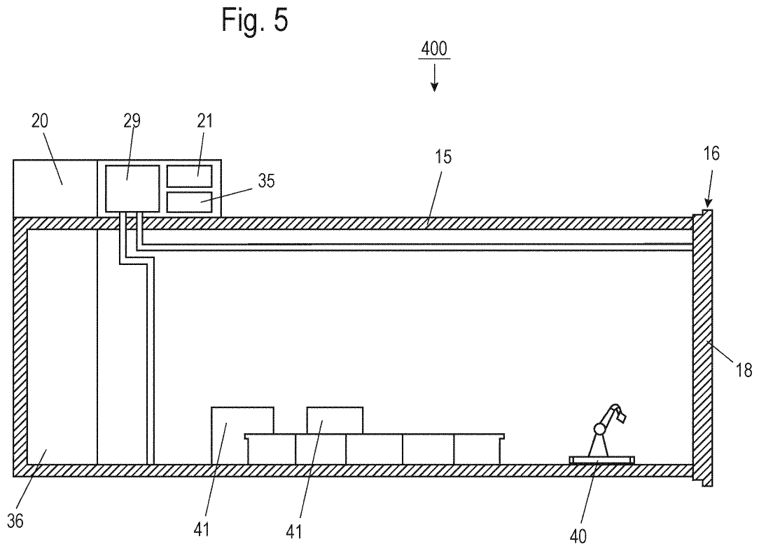

FIG. 5 shows a maintenance unit 300, which may have a lifting mechanism 37 and/or a drive 38 for vertical and horizontal movement. The maintenance unit 300 can have a docking point 16 at one location, in particular at a first end, and a personnel lock 34 at another location, in particular at a second end, and a power supply and management unit 20 as well as insulation 15 inside. The interior can have a conveyor system 37 to convey the storage rack 14 and a robot-like installation 40 for automated harvesting. Additionally, the maintenance unit 300 may include a storage space 41.

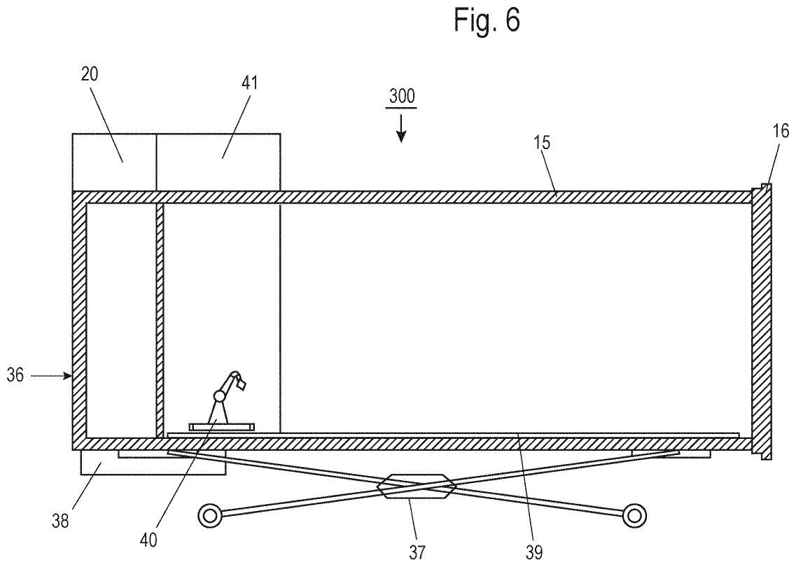

FIG. 6 illustrates a process unit 500, which may include a power supply and management unit 20, a personnel lock 39, a docking station 16, and an internal isolation 15. The power supply and management unit 20 may include a technology side 19, a communication module 21, and/or a reservoir 28. The docking site 16 may include a double door 18. The interior of the process unit 500 may have one or more technical installations, a majority of which are robot-like installations 40, installed therein and a storage area 41 for unprocessed and processed goods.

Although the invention has been illustrated and described in detail by way of preferred embodiments, the invention is not limited by the examples disclosed, and other variations can be derived from these by the person skilled in the art without leaving the scope of the invention. It is therefore clear that there is a plurality of possible variations. It is also clear that embodiments stated by way of example are only really examples that are not to be seen as limiting the scope, application possibilities or configuration of the invention in any way. In fact, the preceding description and the description of the figures enable the person skilled in the art to implement the exemplary embodiments in concrete manner, wherein, with the knowledge of the disclosed inventive concept, the person skilled in the art is able to undertake various changes, for example, with regard to the functioning or arrangement of individual elements stated in an exemplary embodiment without leaving the scope of the invention, which is defined by the claims and their legal equivalents, such as further explanations in the description.

### REFERENCE SIGN

1 Storage container
2 Center segment
3 Interior
4 Substrate
5 Nutrients
6 Development material
7 Wall
8 End segment
9 Mechanical interface
10 Protective layer
11 Barrier layer
12 Supporting layers
13 Limit stop
14 Storage rack
15 Isolation
16 Docking point
17 Maintenance unit
18 Double door
19 Technical compartment
20 Energy supply and management unit
21 Communication module
22 Uninterruptible power supply
23 Light source
24 Air conditioner
25 Ventilation slots
26 Irrigation system
27 Water treatment
28 Reservoir
29 Actuators
30 Nutrient container
31 Mixing chamber
32 Control and evaluation unit
33 Sensors
34 Trays
35 Data storage
36 Personnel lock
37 Lifting mechanism
38 Drive
39 Conveyor system
40 Robot like installation
41 Storage room
100 Growth or propagation station
200 Farm
300 Maintenance unit
400 Processing system
500 Process unit

1-24. (canceled)

25. A storage container, comprising:
an interior space;
a substrate arranged in the interior space, wherein the substrate is stationarily arranged on a wall of the storage container, and wherein the substrate is an absorbent material with a water absorption capacity in the dry state of at least 50 g water/cm³;
a biological living development material arranged in or on the substrate, wherein the storage container is a sealed capsule ensuring an aseptic atmosphere in the interior space;
at least two end segments; and
a middle segment, which is tubular, wherein the substrate and the development material are arranged in the middle segment, and at least a first of the two end segments is arranged opposite the middle segment,
wherein the first of the two end segments has
a lower wall thickness than the middle segment,
a wall material that is more thermally, physically, or chemically susceptible to attack than the middle segment, or
a predetermined separation point.

26. The storage container of claim 25, wherein a material of the substrate comprises at least one porous material, at least one sponge material, or at least one gel-like material in which water is present in stored form.

27. The storage container of claim 25, wherein
the storage container has a gas-filled or vacuumed cavity below the substrate, wherein the gas-filled or vacuumed cavity is bounded by wall sections of the storage container and by the substrate, or
the substrate is arranged as an only water reservoir within the storage container and is positioned with a water content of 30 g water/cm³ in the middle segment of the storage container.

28. The storage container of claim 25, wherein the first of the end segments is a bottom end segment with respect to the development material.

29. The storage container of claim 25, wherein at least a second of the two end segments is a lid-side end segment formed of a gas-selective semi-permeable material, which is CO₂, O₂, N₂, or CH₄-selective.

30. The storage container of claim 25, wherein
a wall of the middle segment consists, at least in regions or completely, of a transparent material which is a transparent, UV-impermeable, thermally insulating and at least partially gas-tight material, or
the storage container has a sterilizable surface of a peroxide- or radical-stable material, which is an ozone-, peracetic acid-, or hydrogen peroxide-stable material, including polyamide, polycarbonate, polyolefin, polyacrylic, polymethacrylic, halogenated ethylene and/or an elastomer such as rubber, halogenated elastomers, and silicones.

31. The storage container of claim 25,
wherein the middle segment has
at least one circumferentially distributed limiting stop,
a plurality of circumferentially distributed limiting stops,
a conical shape, or
a locking device configured to fix the storage container in a stationery and vibration-resistant manner, or
wherein the limiting stop or stops or the locking device is attached to a lower half of the middle segment.

32. An industrial growth or propagation station configured to cultivate development material, the growth and/or propagation station comprising:
a large-capacity ISO container having a sensor-monitored distribution device, configured to provide and monitor an aseptic internal atmosphere with simultaneous setting, monitoring, or maintenance of optimum growth conditions, wherein to ensure the optimum growth conditions the sensor-monitored distribution device is configured to sense and adjust at least one of the following parameters, air pressure, temperature, humidity, and gas exchange rate.

33. The industrial growth or propagation station of claim 32, wherein the sensor-monitored distribution device is further configured to sense and adjust, either alone or in combination with other sensors, at least one of the following parameters
SAL level,
partial gas pressure of a single or a combination of the following gases CO₂, O₂, N₂, He, Ar, O₃, CO, CH₄, ethane, ethene, ethyne, or terpenes,
fertilizer temperature,
wind strength,
wind direction,
sound level or sound frequency sequence,
light intensity,
fertilizer quantity,
fertilizer composition,
pH value, or
conductance.

34. The industrial growth or propagation station of claim 33, wherein the terpenes are single or in combination hemiterpenes, monoterpenes, sesquiterpenes, diterpenes, sesterterpenes, triterpenes, tetraterpenes, polyterpenes, and terpenoids.

35. The industrial growth or propagation station of claim 32, further comprises:
a grow room with an inlet or outlet of a cleaning medium for clean-in-place cleaning or for an ozone clean-in-place medium.

36. The industrial growth or propagation station of claim 32, wherein the large ISO capacity container comprises at least one storage rack configured to arrange one or more storage containers, which comprise
an interior space;
a substrate arranged in the interior space, wherein the substrate is stationarily arranged on a wall of the storage container, and wherein the substrate is an absorbent material with a water absorption capacity in the dry state of at least 50 g water/cm³;
a biological living development material arranged in or on the substrate, wherein the storage container is a sealed capsule ensuring an aseptic atmosphere in the interior space;
at least two end segments; and
a middle segment, which is tubular, wherein the substrate and the development material are arranged in the middle segment, and at least a first of the two end segments is arranged opposite the middle segment,
wherein the first of the two end segments has
a lower wall thickness than the middle segment,
a wall material that is more thermally, physically, or chemically susceptible to attack than the middle segment, or
a predetermined separation point.

37. A cultivation system, comprising:
the growth or propagation station of claim 32;
a maintenance unit; and
a process unit, which is a harvesting unit,
wherein the maintenance unit is connected to the growth or propagation unit to transfer development material or machinery between the growth or propagation station, the maintenance unit, and the process unit, and
wherein the maintenance unit or process unit at least in combination with the growth or propagation station is configured to maintain an aseptic internal atmosphere.

38. A method for cultivating development material, the method comprising:
a) providing the storage container of claim 25;
b) inserting the storage container into a storage rack of a growth or propagation station, which comprises a large-capacity ISO container having a sensor-monitored distribution device, configured to provide and monitor an aseptic internal atmosphere with simultaneous setting, monitoring, or maintenance of optimum growth conditions;
after insertion of the storage container into the storage rack, sterilizing without dismantling an arrangement of storage rack and storage container, and opening the storage container at least at one end;
c) monitoring growth or propagation of the development material in the aseptic internal atmosphere, until detection of a growth or propagation stage for harvesting;
d) using a maintenance unit or a process unit while maintaining the aseptic atmosphere while recovering a biological material containing an active substance from the harvested development material.

39. The method of claim 38, wherein
in step c), to control an atmosphere for growth or propagation, at least one of the parameters are determined, monitored, or adjusted
air pressure,
temperature,
humidity,
light intensity,
gas exchange rate,
fertilizer quantity,
fertilizer composition,
pH value, and
conductance, or
wherein in step c), to control the atmosphere for growth or propagation, at least one of the following parameters are determined, monitored, or adjusted, SAL level, partial gas pressure, fertilizer temperature, wind intensity, wind direction, sound level, or sound frequency sequence.
